(19) United States
(12) Patent Application Publication
(10) Pub. No.: US 2011/0004140 A1
(43) Pub. Date: Jan. 6, 2011

(54) Equine wrap

(76) Inventor: Christina Green, Parker, CO (US)

Correspondence Address: Christina Green, 11245 East Parker Rd, Parker, CO, 80138, US

(21) Appl. No.: 12/459,446

(22) Filed: Jul. 1, 2009

(51) Int. Cl. A61F 13/06

(52) U.S. Cl. 602/62

(57) ABSTRACT

An improved equine wrap comprises a bandage/wrap material, a clue-mark, and an improved fastener. The bandage/wrap material can be any suitable material used, or yet to be used, in the art. Examples include neoprene, elastic bandages known as “Ace bandages,” etc. The present invention provides a clue-mark on the initiating end of a wrap in order to assist the wrapper in applying the wrap in the correct direction. The wrapper need only determine whether the leg to be wrapped is a right leg or a left leg and then follow the instructions given by the clue-mark. The improved fastener comprises a double-sided tab and a double-sided attachment point. Therefore, after application of the wrap, the improved fastener can be utilized regardless of which side of the wrap the wrapper initially chose to face outwards.

### TECHNICAL FIELD

This invention relates generally to the field of bandages and wraps and in particular to an improved limb wrap that can be used for horses, etc.

### BACKGROUND

Leg bandages or “wraps” have been used on horses for years. There are many reasons for their use including protective as well as therapeutic purposes. Wrapping can provide both protection and support for a horse while working, traveling, resting or recovering from an injury. Wraps provide support for tendons and ligaments during strenuous workouts, prevent or reduce swelling after exercise or injury, protect horses' legs from impacts or excessive rubbing, provide a shield for wounds against contamination, and can aid in healing. Furthermore, wraps can also be applied to a horse's tail.

A wrap, or polo wrap, is generally wrapped around a horse's leg and covers some portion of the leg between the knee and the fetlock. Wraps are generally nine to twelve feet in length and four to six inches in width, although other lengths and widths are utilized. Wraps are applied by winding the material around a horse's leg and overlapping each subsequent wind by approximately half the width of the wrap (although this amount varies considerably). Methods of applying wraps also vary depending on the purpose for the wrap, type of wrap, and the person performing the wrapping. However, a standard rule in the application of equine wraps is that a poorly applied wrap or bandage can do more harm than good.

It is essential that the person wrapping a leg (the “wrapper”) use proper techniques. Applied incorrectly, wraps might not only fail to do their job, they can cause discomfort, restrict blood flow and potentially damage tendons and other tissues. For example, in order to prevent tendon damage, a horse's leg should be wrapped from the inside around the front of the leg. This means wrapping clockwise on the horse's right legs and counterclockwise on the horse's left legs. For beginner wrappers, this can be confusing and difficult to remember. However, wrapping in the wrong direction even one time can have serious consequences.

There are many shapes and sizes of wraps currently known in the art, but all have significant deficiencies. As mentioned above, the direction of wrapping is critically important and changes depending on whether the leg being wrapped is a left leg or a right leg. Current wraps do not assist the person applying the wrap in determining the correct direction for wrapping. Because of the time required to re-apply a wrap which has been wound in the wrong direction and the potential harm to a horse if such a wrap is inadvertently overlooked and subsequently left in place, there is a significant need for a wrap that assists the wrapper in applying the wrap in the correct direction.

Many wraps are consistently sized and shaped along their entire length. While some wraps have identical ends, many have a distinct initiating end and a distinct terminating end. For example, an initiating end may have no fastening means attached thereto as the pressure from subsequent layers of the wrap will hold the initiating end in place. However, the terminating end may have an integrated fastening means attached thereto. The fastening means is used to hold the terminating end in place so that the tension and placement of the wrap is maintained. A common fastening means has two parts: a tab which is a strip of hook or loop material (e.g. material sold under the trade mark “VELCRO”) extending out from the terminating end of the wrap, and an attachment point which is a corresponding strip of loop or hook (respectively) material placed on the wrap, some distance back from the terminating end. As the final length of the terminating end of the wrap is wound around the leg, the tab will be automatically located above the attachment point. The person wrapping the leg then simply presses the tab onto the attachment point and the hook and loop materials then engage one another, effectively locking the tab to the attachment point. However, if the person applying the wrap inadvertently turns the wrap over before starting application of the wrap to the leg, the attachment point will end up facing the horse's leg instead of facing outwards so as to accept the tab. In such an instance, the leg will have to be unwrapped, the wrap turned over, and then the leg will need to be rewrapped correctly. This process is time-consuming and aggravating and can lead to poorly wrapped legs. Similar problems occur when using wraps in non-equine-related applications.

Therefore, there is a need for an improved wrap that addresses the above limitations and problems.

### SUMMARY

Embodiments of the present invention described and claimed herein address the foregoing limitations and problems by providing an improved equine wrap. The present invention provides a clue-mark on both sides of the initiating end of a wrap in order to assist the wrapper in applying the wrap in the correct direction. Furthermore, an improved fastening means has been added which can be utilized regardless of which side of the wrap the wrapper chooses to face outwards.

In one embodiment, an equine wrap comprises a bandage/wrap material, a clue-mark, and an improved fastening means. The bandage/wrap material can be any suitable material currently known or yet to be discovered in the art. Examples of suitable materials include neoprene, elastic bandages known as “Ace bandages,” etc.

A clue-mark to assist the wrapper in applying the wrap in the correct direction can be represented in various ways. In one embodiment, the clue-mark comprises a letter “R”, an arrow, and a letter “L”. The “R” is placed near the initiating end of the wrap such that the letter is right-side up and the initiating end terminates after the “R”. The arrow is placed under the “R” and points to the terminating end of the wrap. The “L” is placed under the arrow and is upside-down relative to the “R”. A similar clue-mark is placed on the other side of the wrap, oriented in a similar manner to the initiating and terminating ends.

In one embodiment, the improved fastening means comprises a double-sided tab and a double-sided attachment point. Thus, regardless of which side of the wrap the wrapper chooses to face outwards, the tab and attachment point will be accessible and can secure the wrap. The tab extends out from the terminating end of the wrap and the attachment point is located on the wrap, a short distance from the terminating end. The tab is covered with either hook or loop material (commonly known as “Velcro”) and the attachment point is covered with the corresponding loop or hook material (i.e., if the tab is covered in hook material, then the attachment point is covered with loop material, and vice-versa). It is important to realize that both sides of the tab are covered in the same material. Likewise, both sides of the attachment point are covered in the same material (although the material is opposite that which covers the tab).

In another embodiment, the tab extends out from the attachment point which itself extends out from the terminating end of the wrap. As the tab is wound around the leg, it comes in contact with the attachment point, as in the embodiment above. However, in this embodiment, the attachment point is not located completely on the wrap but extends outwards past the terminating end of the wrap.

The present invention provides many benefits over the prior art. Because the clue-mark clearly details the direction that a wrapper is to apply the wrap for a given Left or Right leg of a horse, the improved wrap significantly reduces occurrences of wrapping in the wrong direction. Furthermore, because the tab and attachment points are in effect double-sided, the wrapper can choose either side of the wrap to face outwards and will still be able to attach the tab to the attachment point once the wrap is in place. Thus, the present invention completely eliminates having to rewrap a leg because the wrong side of the wrap was selected to face outwards; effectively, there is no ‘wrong’ side to the improved wrap. Such a problem occurs when using wraps in the human orthopedic environment and so the present invention could also have applicability there.

### BRIEF DESCRIPTION OF THE DRAWINGS

The aforementioned and other features and objects of the present invention and the manner of attaining them will become more apparent and the invention itself will be best understood by reference to the following descriptions of a preferred embodiment and other embodiments taken in conjunction with the accompanying drawings, wherein:

FIG. 1 illustrates a side view of an exemplary embodiment of an improved wrap in an unrolled state.

FIG. 2 illustrates a perspective view of an exemplary embodiment of an improved wrap in a partially rolled-up state.

FIG. 3 illustrates a side view of an exemplary embodiment of an improved wrap applied to a horse's leg.

FIG. 4 illustrates a side view of another exemplary embodiment of an improved wrap in an unrolled state.

### DETAILED DESCRIPTION

In one embodiment, an equine wrap comprises a wrap having a clue-mark and an improved fastening means. The clue-mark can be placed on one or both sides of the initiating end of a wrap in order to assist the wrapper in applying the wrap in the correct direction. The improved fastening means can be utilized regardless of which side of the wrap the wrapper chooses to face outwards. The bandage/wrap material can be any suitable material currently known, or yet to be known, in the art. Examples of suitable materials include neoprene, elastic bandages known as “Ace bandages,” etc.

A clue-mark to assist the wrapper in applying the wrap in the correct direction can be represented in various ways. In one embodiment, the clue-mark comprises a letter “R”, an arrow, and a letter “L”. The letter “R” is placed near the initiating end of the wrap such that the letter can be read (i.e., it is right-side up) and the initiating end of the wrap comes to an end just to the right of the “R” (after the “R” when it is read from left to right). The arrow is placed under the “R” and points away from the initiating end of the wrap. The “L” is placed under the arrow and is upside-down relative to the “R”. A second clue-mark can be placed on the other side of the wrap, oriented in a manner similar to the first clue-mark relative to the initiating and terminating ends of the wrap.

In one embodiment, the improved fastening means comprises a tab and an attachment point. The tab extends out from the terminating end of the wrap and the attachment point is located on the wrap, a short distance from the terminating end (the attachment point can also extend out from the wrap). The tab is covered with either hook or loop material and the attachment point is covered with the corresponding loop or hook material, respectively. It is important to realize that both sides of the tab are covered in the same material. Likewise, both sides of the attachment point are covered in the same material in order to be able to attach to the tab.

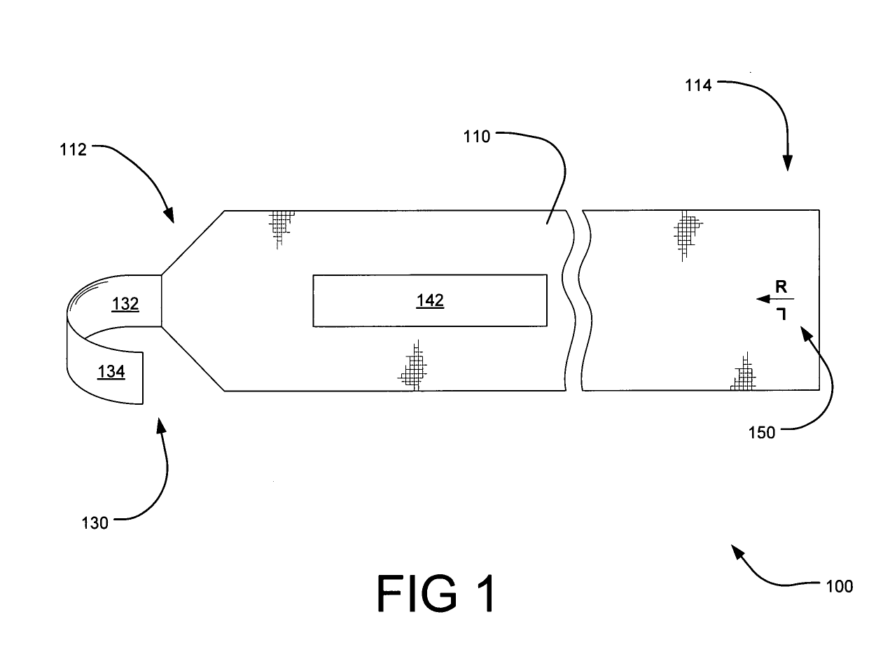

FIG. 1 illustrates a side view of an exemplary embodiment of an improved wrap 100 in an unrolled state. The primary components shown in FIG. 1 include: the wrap body 110, the tab 130, and the attachment point 142. The front side or first side of the wrap body 110 is shown in FIG. 1. The back side or second side of the wrap body 110 is not shown. The wrap body 110 has two ends, an initiating end 114 and a terminating end 112. As shown in FIG. 1, a clue-mark 150 is positioned near the initiating end 114. The initiating end 114 is that end of the wrap body 110 that a wrapper applies to begin the process of wrapping a leg, tail, etc.

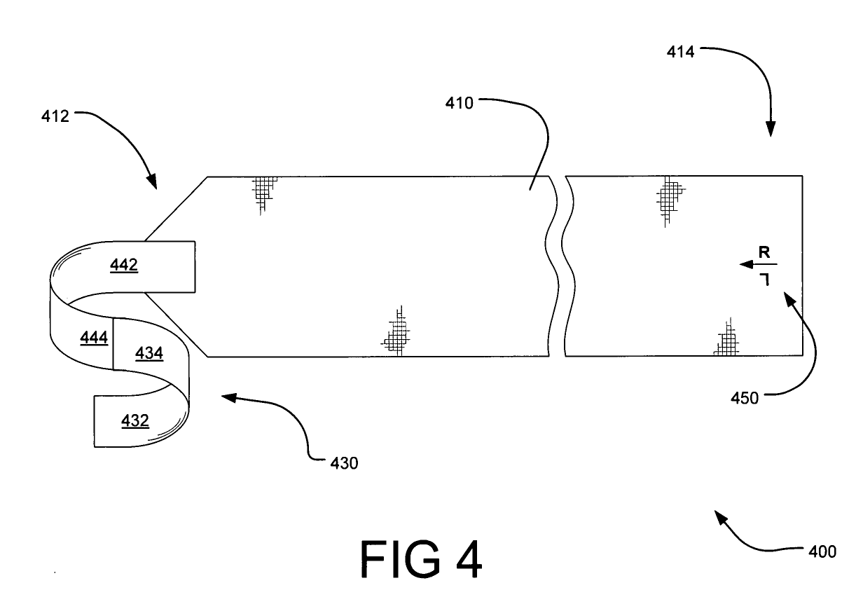

The attachment point 142 is shown on the front side of the wrap body 110. Another attachment point 144 is placed on the back side of the wrap body 110 (not shown in FIG. 1). In other embodiments, the attachment points 142 and 144 are located slightly differently (see FIG. 4). The attachment points 142 and 144 are placed so that the tab 130 can be secured against the attachment points 142 and 144 at the completion of the wrapping process. Thus, regardless of their exact positions, the attachment points 142 and 144 should be located near the terminating end 112 of the wrap body 110.

As shown in FIG. 1, the tab 130 extends out past the terminating end 112 of the wrap body 110 and has two sides: a front side 132 and a back side 134. Both sides 132 and 134 of the tab 130 have hook and loop material attached. In one embodiment, the front side 132 of the tab 130 has hook material facing outwards and the back side 134 of the tab 130 has hook material facing outwards. In this embodiment, the attachment points 142 and 144 would have loop material in order to mate up with the tab 130.

In another embodiment, the front side 132 of the tab 130 has loop material facing outwards and the back side 134 of the tab 130 has loop material facing outwards. In this embodiment, the attachment points 142 and 144 would have hook material in order to mate up with the tab 130.

In yet another embodiment, one side 132 or 134 of the tab 130 has hook material and the other side 134 or 132 has loop material. In this embodiment, the corresponding attachment points 142 and 144 have opposing loop material and hook material, respectively, in order to mate up with the corresponding sides 132 and 134 of the tab 130. It is conceivable that both hook material and loop material can be located on both sides 132 and 134 of the tab 130 as well as on both attachment points 142 and 144.

In the embodiment in FIG. 1, only one clue-mark 150 is displayed. However, a corresponding clue-mark 152 is located on the back side of the wrap body 110. Clue-marks 150 and 152 are used by the wrapper to assist in applying the improved wrap 100 in the correct direction. In one embodiment, the clue-marks 150 and 152 comprise a letter “R”, an arrow, and a letter “L”. The first clue-mark 150 can be seen on the front side of the wrap body 110 in FIG. 1. The letter “R” is placed near the initiating end 114 of the wrap body 110 such that the letter can be easily read by the wrapper (i.e., it is right-side up) and the initiating end 114 of the wrap body 110 comes to an end just to the right of the “R” (after the “R” when it is read from left to right). The arrow is placed under the “R” and points away from the initiating end 114 of the wrap body 110 towards the terminating end 112. In other embodiments, the arrow can be placed above or otherwise near the “R”. Regardless of its exact position, it is important that the arrow point in the same direction relative to the “R”. The “L” is placed under the arrow and is upside-down relative to the “R”. In other embodiments, the “L” is placed elsewhere. Regardless of its exact position, it is important that the “L” be oriented upside-down relative to the “R”. On the back side of the wrap body 110, a second clue-mark 152 can be placed (not shown in FIG. 1). The clue-mark 152 on the back side of the wrap110 is oriented in a manner similar to the first clue-mark 150 relative to the initiating end 114 and the terminating end 112 of the wrap body 110.

The orientation of the clue-marks 150 and 152 is important so as to inform a wrapper of the correct direction to apply the improved wrap 100. For example, a wrapper would begin application of an improved wrap 100 by unrolling the initiating end 114 from a rolled-up improved wrap 100. The wrapper then determines which side of the horse is to be wrapped. If the leg to be wrapped is on the horse's left side, then the wrapper places the initiating end 114 of the wrap 100 on the horse's leg such that the letter “L” on the clue-mark 150 or 152 is right-side up (i.e., the wrapper can read the letter). Once placed, the wrapper then wraps the leg by unwinding the roll and winding the wrap 100 around the horse's leg in the direction indicated by the arrow portion of the clue-mark 150 or 152. Similarly, if the leg to be wrapped is on the horse's right side, then the wrapper places the initiating end 114 of the wrap 100 on the horse's leg such that the letter “R” on the clue-mark 150 or 152 is right-side up (i.e., the wrapper can read the letter). Once placed, the wrapper then wraps the leg by unwinding the roll and winding the wrap 100 around the horse's leg in the direction indicated by the arrow portion of the clue-mark 150 or 152. Thus, the clue-marks 150 and 152 ensure that the improved wrap 100 is always wound clockwise on a horse's right legs and counter-clockwise on a horse's left legs.

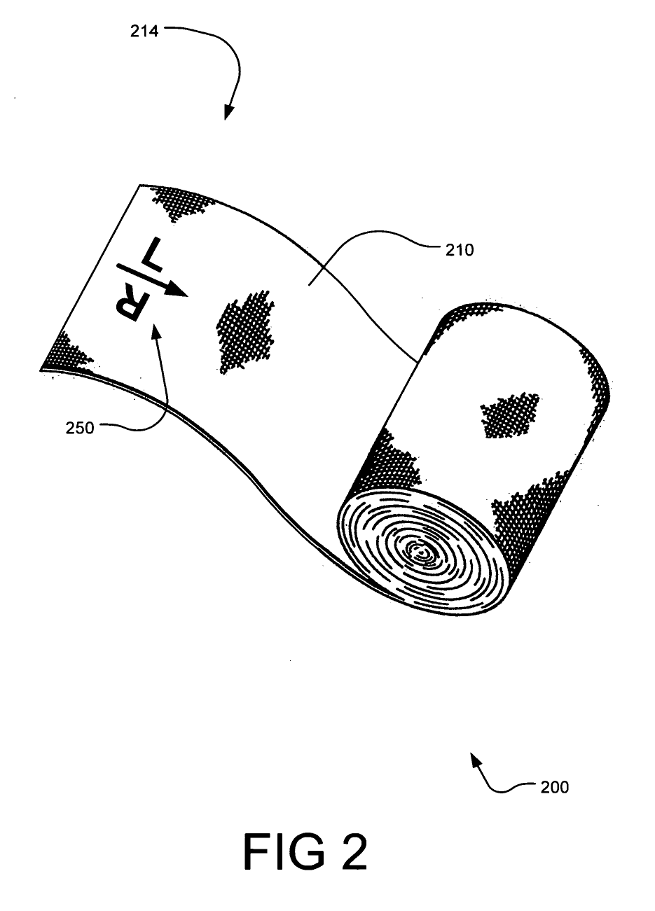

FIG. 2 illustrates a perspective view of an exemplary embodiment of an improved wrap 200 in a partially rolled-up state. The components shown in FIG. 2 include: the wrap body 210, the initiating end 214, and the clue-mark 250. Wraps 200 are commonly stored in a rolled-up state to keep them orderly and to assist in their application (as applying ten-plus feet of loose wrap 200 can be difficult).

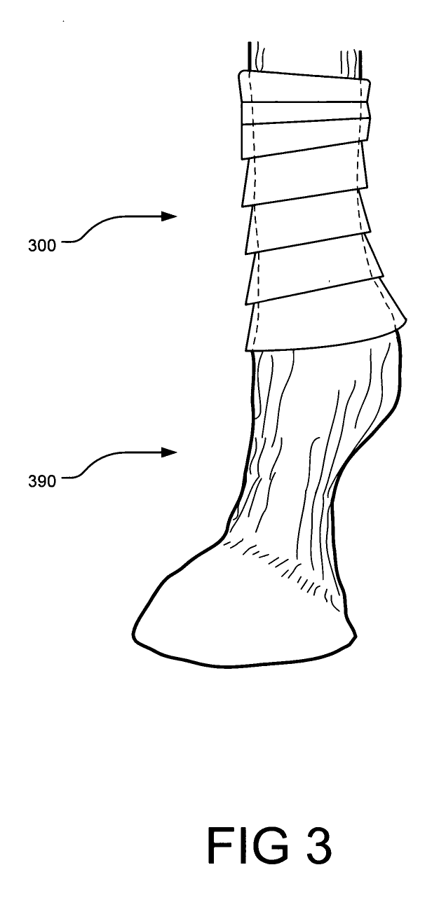

FIG. 3 illustrates a side view of an exemplary embodiment of an equine wrap 300 applied to a horse's leg 390. It is preferred that the wrapper place the initiating end 114 (not shown in FIG. 3, see FIG. 1) of the wrap 300 at some point above the lowest point that needs to be wrapped and then wrap downwards, overlapping one-half to two-thirds of the width of the wrap 300 as he or she progresses. Appropriate tension on the wrap 300 should be kept at all times. Once the wrapper reaches the lowest point on the leg that needs to be wrapped, he or she begins wrapping back upwards towards the knee or hock wrapping over the initiating end and continuing upwards. As the terminating end 112 (not shown in FIG. 3, see FIG. 1) of the wrap 300 is reached, the wrapper simply attaches one side 132 or 134 of the tab 130 to the attachment point 142 or 144. The terminating end 112, front side 132, back side 134, tab 130 and attachment points 142 and 144 are not shown in FIG. 3, see FIG. 1.

FIG. 4 illustrates a side view of another exemplary embodiment of an improved wrap 400 in an unrolled state. The primary components shown in FIG. 4 include: the wrap body 410, the tab 430, and the attachment points 442 and 444. The front side or first side of the wrap body 410 is shown in FIG. 4. The back side or second side of the wrap body 410 is not shown. The wrap body 410 has two ends, an initiating end 414 and a terminating end 412. As shown in FIG. 4, a clue-mark 450 is positioned near the initiating end 414. The initiating end 414 is that end of the wrap body 410 that a wrapper applies to begin the process of wrapping a leg, tail, etc.

The attachment point 442 is shown extending out from the front side of the wrap body 410. A second attachment point 444 is placed on the back side of the first attachment point 442. In other embodiments, the attachment points 442 and 444 are located slightly differently (see FIG. 1). The attachment points 442 and 444 are placed so that the tab 430 can be secured against the attachment points 442 and 444 at the completion of the wrapping process.

As shown in FIG. 4, the tab 430 extends out past the attachment points 442 and 444 and has two sides: a front side 432 and a back side 434. Both sides of the tab 430 have hook and loop material attached. In one embodiment, the front side 432 of the tab 430 has hook material and the back side 434 of the tab 430 has hook material. In this embodiment, the attachment points 442 and 444 would have loop material in order to mate up with the tab 430.

In another embodiment, the front side 432 of the tab 430 has loop material and the back side 434 of the tab 430 has loop material. In this embodiment, the attachment points 442 and 444 would have hook material in order to mate up with the tab 430.

In yet another embodiment, one side of the tab 430 has hook material and the other side has loop material; in this embodiment, the corresponding attachment points 442 and 444 have opposing loop material and hook material, respectively, in order to mate up with the corresponding sides 434 and 432 of the tab 430. For example, if the front side 432 of the tab 430 has hook material, then the back side 434 of the tab 430 has loop material; and the front attachment point 442 would have hook material so that the loop material on the back side 434 of the tab would secure with the hook material on the front attachment point 442. Similarly, if the wrap 400 was flipped over before application, then the back attachment point 444 would need to have loop material in order to secure with the hook material on the front side 432 of the tab 430.

The above specification, examples and data provide a description of the structure and use of exemplary embodiments of the described articles of manufacture and methods. Many embodiments can be made without departing from the spirit and scope of the invention.

What is claimed is:

1. A wrap that can be used on a horse adapted to be wrapped around a portion of a body in overlapping convolutions, comprising:
a wrap body having an initiating end and a terminating end;
a double-sided tab attached to the wrap body in proximity to the terminating end;
a double-sided attachment point attached to the wrap body in proximity to the terminating end and positioned so as to removably fasten to the tab once the wrap is applied; and
a clue-mark positioned on the wrap body in proximity to the initiating end and oriented so as to assist in applying the wrap in a correct manner.

2. The wrap of claim 1, wherein the double-sided tab is at least partially covered with a hook and loop material.

3. The wrap of claim 1, wherein the double-sided attachment point is at least partially covered with a hook and loop material.

4. The wrap of claim 2, wherein the double-sided attachment point is at least partially covered with a hook and loop material.

5. The wrap of claim 4, wherein the hook and loop material on the tab comprises at least hook material and the hook and loop material on the attachment point comprises at least loop material.

6. The wrap of claim 4, wherein the hook and loop material on the tab comprises at least loop material and the hook and loop material on the attachment point comprises at least hook material.

7. The wrap of claim 4, wherein the hook and loop material on the tab comprises both loop material and hook material.

8. The wrap of claim 4, wherein the hook and loop material on the attachment point comprises both hook material and loop material

9. A wrap that can be used on a horse adapted to be wrapped around a portion of a body in overlapping convolutions, comprising:
a wrap body having an initiating end and a terminating end;
a double-sided attachment point attached to the wrap body in proximity to the terminating end;
a double-sided tab attached to the attachment point and positioned so as to removably fasten to the attachment point once the wrap is applied; and
a clue-mark positioned on the wrap body in proximity to the initiating end and oriented so as to assist in applying the wrap in a correct manner.

10. The wrap of claim 9, wherein the double-sided tab is at least partially covered with a hook and loop material.

11. The wrap of claim 9, wherein the double-sided attachment point is at least partially covered with a hook and loop material.

12. The wrap of claim 10, wherein the double-sided attachment point is at least partially covered with a hook and loop material.

13. The wrap of claim 12, wherein the hook and loop material on the tab comprises at least hook material and the hook and loop material on the attachment point comprises at least loop material.

14. The wrap of claim 12, wherein the hook and loop material on the tab comprises at least loop material and the hook and loop material on the attachment point comprises at least hook material.

15. The wrap of claim 12, wherein the hook and loop material on the tab comprises both loop material and hook material.

16. The wrap of claim 12, wherein the hook and loop material on the attachment point comprises both loop material and hook material.

16. A method for applying a wrap to a leg of a horse, the method comprising:
determining whether the leg to be wrapped is one of the horse's left legs or right legs;
placing an initiating end of the wrap on the leg with a clue-mark displaying in a correct orientation based on whether the leg is a right leg or a left leg;
wrapping the leg in a direction indicated by the clue-mark;
securing a terminating end of the wrap by removably affixing a tab to an attachment point.

17. The method of claim 16, wherein the clue-mark comprises at least an “R”, an “L”, and an arrow; and the placing step further comprises orienting the “R” in an upright orientation if the leg is a right leg or orienting the “L” in an upright orientation if the leg is a left leg.

18. The method of claim 17, wherein the direction indicated by the clue-mark is determined by the direction in which the arrow is pointing.

19. The method of claim 16, wherein the step of securing the terminating end comprises pressing a hook and loop material attached to the tab to a hook and loop material attached to the attachment point.

20. The method of claim 18, wherein the step of securing the terminating end comprises pressing a hook and loop material attached to the tab to a hook and loop material attached to the attachment point.
